(19) United States
(12) Patent Application Publication
(10) Pub. No.: US 2021/0236314 A1
(43) Pub. Date: Aug. 5, 2021

(54) BALLOON CATHETERS, SYSTEMS AND METHODS

(71) Applicant: W. L. Gore & Associates, Inc., Newark, DE (US)

(72) Inventors: Jane K. Bohn, Flagstaff, AZ (US); Richard M. Bruchman, Flagstaff, AZ (US); Christopher Hyzin, Flagstaff, AZ (US); Mark A. Rittgers, Phoenix, AZ (US)

(21) Appl. No.: 17/050,315

(22) PCT Filed: Apr. 23, 2019
(86) PCT No.: PCT/US2019/028738

§ 371 Date: Oct. 23, 2020

Related U.S. Application Data
(60) Provisional application No. 62/661,942, filed on Apr. 24, 2018.

(51) Int. Cl. A61F 2/958; A61M 25/10

(52) U.S. Cl. CPC A61F 2/958; A61M 2025/1084; A61M 2025/1081; A61M 25/10

(57) ABSTRACT

Various aspects of the present disclosure are directed toward apparatuses, systems, and methods that include an implantable medical device having a nominal device diameter and a delivery catheter including a balloon.

### CROSS-REFERENCE TO RELATED APPLICATION

This application claims the benefit of Provisional Application No. 62/661,942, filed Apr. 24, 2019, which is incorporated herein by reference in its entirety for all purposes.

### TECHNICAL FIELD

The present invention generally relates to balloons for balloon catheters. More specifically, the present invention relates to balloons having rather small delivery profiles and delivery diameters when compressed onto delivery catheters.

### BACKGROUND

Balloon catheters can be used in a variety of medical settings, including for use in treating vascular disease. Balloon catheters can be used for angioplasty treatment as part of a minimally-invasive endovascular procedure. In addition, balloon catheters are used to dilate tissue (e.g., vascular structures), expand medical devices (e.g., stents or stent grafts) deployed in vessels or other openings, or combinations thereof in association with an endoluminal medical procedure. Balloon catheters can also be used for occlusion purposes (e.g., to temporarily block vasculature). To facilitate delivery to a variety of treatment locations, it is desirable for the balloon catheter to have a small balloon diameter (e.g., delivery diameter) when the balloon is in its compressed state. Current compression techniques involve folding, pleating, and/or scrunching balloons to reduce the balloons' compressed diameters while maintaining overall size/surface area upon inflation.

Balloon wall thicknesses can be selected based upon a desired rated burst pressure and final device profile. The rated burst pressure determines the amount of internal pressure the balloon can withstand before rupturing and is an important variable in balloon catheter efficacy and safety. Therefore, the compressed diameter of a balloon may be tied to the wall thickness of the balloon and rated burst pressure the balloon is capable of withstanding. It would be desirable for balloons to have increasingly smaller compressed diameters while being able to withstand the same or similar rated burst pressures.

### SUMMARY

Various examples relate to balloon catheters and catheter systems for use in a variety of different body passageways and body lumens. In particular, various examples relate to balloon catheters and implantable medical devices having a small delivery profile capable of reaching relatively small body lumens prior to expansion. The catheter system generally includes a delivery catheter, an implantable medical device, and a balloon.

According to one example (“Example 1”), a system for delivery of an implantable medical device includes an implantable medical device having a delivery diameter and a nominal device diameter. The implantable medical device is capable of expanding from a delivery diameter to the nominal device diameter, wherein the nominal device diameter is larger than the delivery diameter. The system also includes a delivery catheter including a balloon. The implantable medical device is mounted on the balloon. The balloon has a nominal balloon diameter that is at least 3.5% larger than the nominal device diameter.

According to another example (“Example 2”) further to Example 1, the balloon has a nominal balloon diameter at a nominal inflation pressure of less than about 8 atm when the implantable medical device is mounted on the balloon.

According to another example (“Example 3”) further to any of Examples 1 and 2, the balloon has a nominal balloon diameter that is from 3.5% to 10% larger than the nominal device diameter.

According to another example (“Example 4”) further to any of Examples 1 to 3, the balloon has a nominal balloon diameter that is from 5% to 10% larger than the nominal device diameter.

According to another example (“Example 5”) further to any of Examples 1 to 4, the balloon has a nominal balloon diameter from 4 mm to 15 mm.

According to another example (“Example 6”) further to any of Examples 1 to 5, the balloon has a nominal balloon diameter of about 7.5 mm.

According to another example (“Example 7”) further to any of Examples 1 to 6, the system also includes a cover that restricts expansion of the balloon. The cover is disposed over at least a portion of the balloon.

According to another example (“Example 8”) further to Example 7, the cover includes an elastomer.

According to another example (“Example 9”) further to any of Examples 7 and 8, the cover is distensible in a radial and an axial direction.

According to another example (“Example 10”) further to any of Examples 7 to 9, the cover includes serpentine fibrils.

According to another example (“Example 11”) further to any of Examples 7 to 10, the cover is radially distensible up to a stop point that restricts expansion of the balloon at a relatively constant inflation pressure.

According to another example (“Example 12”), a balloon catheter includes a catheter and a balloon mounted on the catheter. The balloon has a nominal inflation pressure and a nominal balloon diameter. The balloon catheter also includes a medical device mounted on the balloon. The medical device has a nominal device diameter. The nominal balloon diameter of the balloon is at least about 5% greater than the nominal device diameter. The nominal inflation pressure of the balloon is about 6 atm when the medical device is mounted on the balloon.

According to another example (“Example 13”), a catheter system includes a balloon having a rated burst pressure and a double wall thickness less than about 60 μm. The balloon is coupled to a catheter. The catheter system also includes a medical device positioned along at least a portion of the balloon. The medical device has a nominal device diameter. A ratio of a pressure of the balloon at the nominal device diameter to the rated burst pressure of the balloon is less than about 0.8.

According to another example (“Example 14”) further to Example 13, the balloon and the medical device are configured to be delivered through a diameter of less than about 2.5 mm when the medical device is positioned along at least a portion of the balloon.

According to another example (“Example 15”), a system for delivery of an implantable medical device includes a balloon having a length. The system also includes a cover disposed over at least a portion of the balloon. The system also includes an implantable medical device disposed over at least a portion of the cover. The cover elastically lengthens during radial expansion of the balloon from a compressed state to an expanded state and imparts a longitudinal lengthening force to the implantable medical device.

According to another example (“Example 16”) further to Example 15, the cover reduces foreshortening of the implantable medical device.

According to another example (“Example 17”) further to any of Examples 16 and 17, the implantable medical device foreshortens less than 20% as compared to the length of the balloon when in a compressed state.

According to another example (“Example 18”), a method for assembling a catheter system includes selecting a balloon catheter with a balloon having a nominal balloon diameter that is at least 5% larger than a nominal device diameter of an implantable medical device. The method also includes mounting the implantable medical device over at least a portion of the balloon. The method also includes compressing the implantable medical device and the balloon into a delivery diameter. Upon expansion of the balloon to the nominal device diameter, a nominal inflation pressure of the balloon is less than or equal to a rated burst pressure of the balloon.

According to another example (“Example 19”) further to Example 18, the method also includes placing a cover over the balloon. The method also includes mounting the implantable medical device over the cover such that the cover imparts a longitudinal lengthening force to the medical device upon inflation of the balloon.

According to another example (“Example 20”) further to any of Examples 18 and 19, the delivery diameter is from 1.5 mm to 3 mm.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 shows a catheter system for delivering an implantable medical device, according to some embodiments.

FIG. 2 shows a close-up view of the balloon and device maintained on a delivery catheter, according to some embodiments.

FIG. 3 shows a nominal balloon diameter and a nominal device diameter, according to some embodiments.

FIG. 4 shows a balloon maintained at an expanded diameter by a cover, according to some embodiments.

FIG. 5 is a cutaway view of a proximal portion of a pleated and folded catheter system, according to some embodiments.

FIG. 6 is a cutaway view of a proximal portion of a non-pleated catheter system, according to some embodiments.

### DETAILED DESCRIPTION

Various aspects of the present disclosure are directed toward balloon catheters and catheter systems for use in a variety of different body passageways and body lumens. In particular, the disclosure relates to balloon catheters and implantable medical devices having a small delivery profile capable of reaching relatively small body lumens prior to expansion.

Expandable medical devices generally have a delivery profile, or delivery configuration with a delivery diameter and an expanded configuration with an expanded diameter (also known as a nominal device diameter). The nominal device diameter may correspond, for example, to the diameter of the body lumen at the desired treatment location. A balloon may be used to expand the expandable medical device from the delivery configuration to the expanded configuration. Similar to the expandable medical device, the balloon also includes a delivery profile with a delivery diameter and an expanded configuration with an expanded diameter.

In certain instances, the size of the delivery profile/configuration of the balloon may depend on various parameters of the balloon, such as the size (length, diameter, and/or thickness). Generally, balloons of smaller sizes (e.g., having a smaller length and/or diameter) are capable of achieving smaller delivery profiles as compared to balloons of larger sizes. However, smaller balloons may have less inflation capability (e.g., available inflation pressure, inflation force, expanded/inflated diameter) as compared to balloons of larger sizes. Thus, a smaller balloon may not be capable of expanding a device to a nominal device diameter that could be expanded by a comparatively larger sized balloon. The balloon catheters and catheter systems discussed herein include balloons capable of achieving a small delivery profile while also retaining the ability to expand a device from a delivery diameter to a nominal device diameter that would previously have required a balloon having a larger profile.

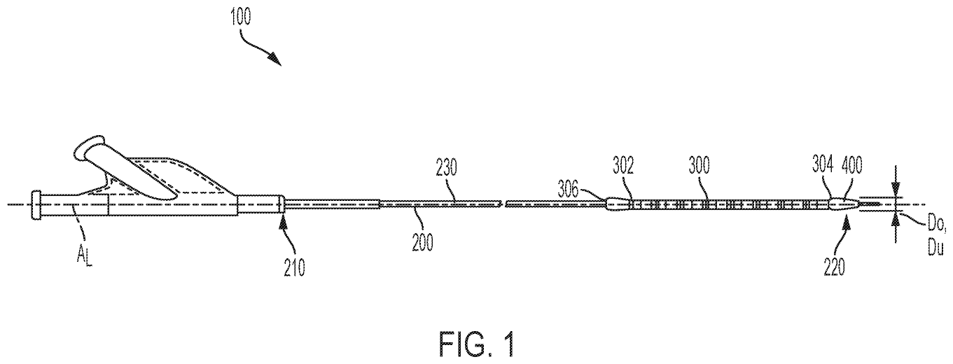

FIG. 1 shows a catheter system 100 for delivering an implantable medical device, according to some embodiments. According to various examples, the system 100 includes a delivery catheter 200, an implantable medical device 300, and a balloon 400. The implantable medical device 300 is maintained over at least a portion of the balloon 400 at a delivery diameter for placement at a desired treatment location within a body of a patient. Although the catheter system 100 is shown configured for delivering an implantable medical device, the catheter system 100 may additionally or alternatively be configured as an occlusion catheter, a drug delivery catheter, or a dilation catheter, as desired. Thus, in some examples, the catheter system 100 may not include the implantable medical device 300.

As shown in FIG. 1, the delivery catheter 200 includes a proximal end 210, a distal end 220, and a lumen 230 extending along a longitudinal axis A_{L} from the proximal end 210 to the distal end 220. In some embodiments, the catheter 200 may be a dual lumen or double lumen catheter. As used herein, the terms “dual lumen catheter” and/or “double lumen catheter” can be defined as a catheter having two lumens or channels for, in some examples, inflow and outflow of fluid.

The delivery catheter 200 may have a length suitable to reach a desired treatment location in the patient's body (e.g., for delivering the implantable medical device 300 to the desired treatment location). For example, the delivery catheter 200 may have a length between about 80 cm and about 140 cm, although other lengths are contemplated and may depend on a variety of factors, including the desired treatment location.

The delivery catheter 200 can include a variety of conventional medical grade materials such as nylon, polyacrylamide, polycarbonate, polyethylene, polyformaldehyde, polymethylacrylate, polypropylene, polytetrafluoroethylene, polytrifluorochlorethylene, polyvinylchloride, polyurethane, elastomeric organosilicon polymers, Pebax® polyether block amide, and metals such as stainless steel and nitinol.

The implantable medical device 300, referred to herein simply as the device 300 may be maintained at or near the distal end 220 of the delivery catheter 200. However, the device 300 can also be maintained at other locations between the proximal end 210 and the distal end 220 along the longitudinal axis A_{L} of the delivery catheter 200 as desired. The device 300 has a first device end 302, a second device end 304, lumen 306 extending along the longitudinal axis A_{L} from the first device end 302 to the second device end 304, a delivery diameter Dₒ and a nominal device diameter D_{d}.

In some embodiments, the delivery diameter Dₒ is sized such that the delivery system 100 may be delivered to the desired treatment location. For example, the delivery diameter Dₒ may be from 1 mm to 5 mm or from 2 mm to 3 mm, although a variety of diameters are contemplated. The delivery diameter Dₒ may also be sized such that the delivery system 100 can be passed through an introducer sheath (not shown) before being placed in the patient's body.

The device 300 may be balloon expandable from the delivery diameter Dₒ to the nominal device diameter D_{d} by inflating or expanding the balloon 400. The nominal device diameter D_{d}, also known as an expanded device diameter, refers to the diameter of the device 300 when the device 300 is in an expanded configuration. In some examples, the nominal device diameter D_{d} is the diameter of the device 300 after inflation of the balloon 400 to a certain desired pressure. The nominal device diameter D_{d} may vary depending on the desired treatment location and/or the anatomy of the patient. In some examples, the nominal device diameter D_{d} corresponds to a diameter at which the device exhibits a sharp increase in resistance to further expansion (e.g., a point at which further expansion under the same radial expansion force ceases). In some embodiments, the nominal device diameter D_{d} is such that the device 300 fits within a natural and/or artificial lumen within the patient's body. For example, the nominal device diameter D_{d} may be from 4 mm to 30 mm, 4 mm to 12 mm, 5 mm to 11 mm, 8 mm to 16 mm, 8 mm to 12 mm, or from 8 mm to 10 mm, although a variety of dimensions are contemplated.

In some embodiments, the device 300 may be a stent, a graft, a stent-graft, or any other expandable, implantable medical device. As is known in the art, the device 300 can be made of a variety of biocompatible materials, including one or a combination of materials. In some embodiments, the device 300 may include steel, such as stainless steel or another alloy, a shape memory material such as nitinol, or a non-metallic material such as a polymeric material. Suitable biocompatible polymeric materials may include, for example, polytetrafluoroethylene (ePTFE), polyester, polyurethane, fluoropolymers, such as perfluoroelastomers and the like, polytetrafluoroethylene, silicones, urethanes, ultra-high molecular weight polyethylene, and aramid fibers, among others.

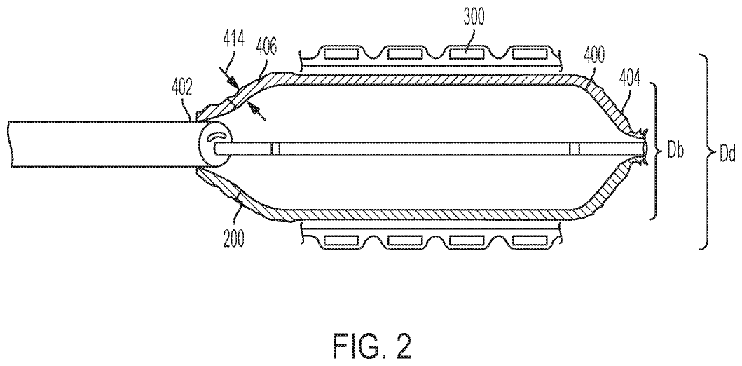

FIG. 2 shows a close-up view of the device 300 maintained over the balloon 400 on the delivery catheter 200. As shown, the balloon 400 may be maintained directly over a portion of the delivery catheter 200. In some embodiments, the balloon 400 may be maintained at or near the distal end 220 of the delivery catheter 200. However, as discussed above, the balloon 400 can be maintained at any location between the proximal end 210 and the distal end 220 of the delivery catheter 200 as desired.

The balloon 400 includes a first balloon end 402, a second balloon end 404, and a wall 406 extending between the first balloon end 402 and the second balloon end 404 to form a lumen 408. In various embodiments, the wall 406 may be a single wall or a double wall. In some embodiments, a single-walled balloon may have a wall thickness from about 15 μm to 100 μm, from 20 μm to 60 μm, or from 25 μm to 45 μm as desired. Double-walled balloons may include, for example, two walls 406 or two layers arranged concentrically. In such cases, the wall thickness is measured as the width or thickness of both walls in combination. In some embodiments, a double walled balloon may also have a wall thickness from about 15 μm to 100 μm, from 20 μm to 60 μm, or from 25 μm to 45 μm, although a variety of dimensions are contemplated.

The balloon 400 is configured to expand to a maximum burst pressure diameter Dₘₐₓ when a rated burst pressure is applied. In some embodiments, the maximum burst pressure diameter Dₘₐₓ of the balloon 400 may depend on the thickness of the wall 406 of the balloon 400. As used herein, “rated burst pressure” can be defined as a pressure at which, with at least a 95% confidence, 99.9% of all balloons will not burst. The “maximum burst pressure diameter,” or labeled burst pressure diameter, is the maximum diameter achievable by the balloon without exceeding the rated burst pressure. In some examples, the rated burst pressure of the balloon 400 is from 8 atm to 20 atm, from 12 atm, to 18 atm, or from 14 atm to 16 atm, although a variety of pressures are contemplated and may depend on a variety of factors including the size, length, and/or wall thickness of the balloon. For reference, rated burst pressure can be determined according to ASTM/ISO standards, such as BS EN ISO 10555-4, for example.

In some embodiments, the balloon 400 is maintained on the delivery catheter 200 at an unexpanded diameter Dᵤ, otherwise referred to herein as a delivery profile or delivery diameter, such that the delivery system 100 may be delivered intraluminally within the body of a patient. In some examples, the unexpanded diameter Dᵤ is such that the delivery system 100 can be passed through an introducer sheath (not shown) before entering the patient's body. For example, the unexpanded diameter Dᵤ may be between about 2 mm and 4 mm, between about 2 mm and 3 mm, or less than 2 mm such as about 1.6 or 1.3 mm or less.

In some embodiments, the balloon 400 is expandable between the unexpanded diameter Dᵤ (e.g., unexpanded configuration) and a nominal balloon diameter Dₙ (e.g., expanded configuration) when a nominal inflation pressure is delivered to the balloon 400. In some examples, the nominal balloon diameter Dₙ is reached when the balloon 400 exhibits a sharp increase in resistance to further expansion at a constant inflation pressure. In some embodiments, this may be the diameter at which further expansion of the balloon 400 would require a user to impart pressure that exceeds the rated burst pressure of the balloon 400. In various examples, the nominal inflation pressure may be from 6 atm to 15 atm, from 8 atm to 15 atm, from 6 atm to 10 atm, from 6 atm to 8 atm, or from 6 atm to 7 atm. These nominal inflation pressures may correspond to various nominal balloon diameters Dₙ. In some embodiments, the nominal balloon diameter Dₙ may be from 4 mm to 15 mm, from 5 mm to 12 mm, from 6 mm to 9 mm or from 6 mm to 7 mm when the balloon 400 is maintained on the delivery catheter 200. For example, the nominal inflation pressure may be from 6 atm to 10 atm when the nominal balloon diameter Dₙ is from 9 mm to 16 mm, or from 11 atm to 16 atm when the nominal balloon diameter Dₙ is from 4 mm to 8 mm.

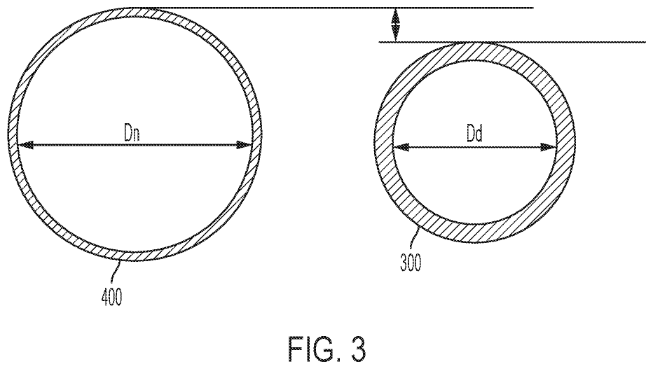

FIG. 3 shows the nominal balloon diameter Dₙ and the nominal device diameter D_{d} according to some embodiments. In some embodiments, the nominal balloon diameter Dₙ may be greater than or equal to the nominal device diameter D_{d}, as described above. In some examples, the nominal balloon diameter Dₙ may be at least 3.5% greater than the nominal device diameter D_{d}, as shown in FIG. 3. In other examples, the nominal balloon diameter Dₙ may be 5%, 6.5%, 7%, or 10% or more greater than the nominal device diameter D_{d}. This enables the device to be sufficiently inflated to the nominal device diameter D_{d}.

In some embodiments, the balloon 400 is expandable between the unexpanded diameter Dᵤ and a maximum burst pressure diameter Dₘₐₓ when the rated burst pressure is delivered to the balloon 400. As discussed above, the balloon 400 may have a rated burst pressure from 8 atm to 20 atm, from 11 atm to 18 atm, or from 12 atm to 16 atm. In some embodiments, the rated burst pressure may be higher than the nominal inflation pressure. For example, the nominal inflation pressure may be less than or equal to the rated burst pressure. In some examples, the nominal inflation pressure may be at least 10% less than the rated burst pressure, at least 15% less than the rated burst pressure, or from 15% to 40% less than the rated burst pressure. In other examples, a ratio of the nominal inflation pressure of a balloon with a stent to the rated burst pressure of a balloon with a stent may be about 0.5, 0.6, 0.7, 0.8, or 0.9. This ratio may allow the balloon 400 to have a higher safety factor as compared to a balloon 400 that does not have characteristics as described above. As discussed above, the rated burst pressure determines the amount of internal pressure the balloon can withstand before rupturing and is an important variable in balloon catheter efficacy and safety.

As used herein, the term “safety factor” may be defined as a ratio of the maximum burst pressure diameter Dₘₐₓ to the nominal balloon diameter Dₙ. For example, a lower ratio of the maximum burst pressure diameter Dₘₐₓ to the nominal balloon diameter Dₙ imparts a higher safety factor or, in other terms, a lower probability of the balloon 400 rupturing when inflated to the nominal balloon diameter Dₙ, as compared to a balloon 400 with a higher ratio of the maximum burst pressure diameter Dₘₐₓ to the nominal balloon diameter Dₙ.

The balloon 400 can include a variety of suitable materials depending on desired expansion characteristics of the balloon 400. In some embodiments, the balloon 400 can include, for example, a non-compliant, generally inelastic balloon. In such embodiments, the balloon 400 can include a material that is configured to allow the balloon 400 to expand to a desired diameter upon sufficient pressurization and remain at or near the desired diameter under further pressurization until a certain burst pressure is reached. For example, suitable materials may include nylon, polyethylene, polyethylene terephthalate (PET), polycaprolactam, polyesters, polyethers, polyamides, polyurethanes, polyimides, ABS copolymers, polyester/polyether block copolymers, ionomer resins, liquid crystal polymers and rigid rod polymers.

In some embodiments, the balloon 400 can include a compliant, relatively elastic balloon. For example, the balloon 400 can comprise a material configured to allow the balloon 400 to continually increase in diameter as the pressure to the balloon 400 is increased. Such materials include, for example, polyurethanes, latex, and elastomeric organosilicone polymers such as polysiloxanes.

In some embodiments, the balloon 400 may include a semi-compliant balloon such that the balloon 400 exhibits both compliant and non-compliant attributes. Although described in connection with compliant and non-compliant embodiments, any material or configuration that allows the balloon 400 to inflate in a desired manner is within the scope of the present disclosure.

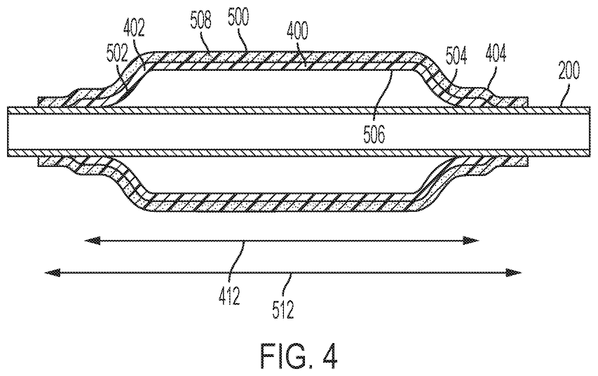

FIG. 4 shows a balloon maintained at an unexpanded diameter by a cover, according to some embodiments. As shown, the balloon 400 is at least partially maintained at the unexpanded diameter Dᵤ by the cover 500. The cover 500 may be maintained over all or a portion of the balloon 400 and may be configured to maintain, constrain, or otherwise restrain the balloon 400 in its unexpanded configuration. In some examples, the balloon 400 is constrained underneath the cover 500, with the cover 500 covering at least a portion, or all of the working length of the balloon. The “working length” of the balloon refers to the portion of the balloon configured to expand and be utilized as part of a medical procedure.

The cover 500 has a first cover end 502, a second cover end 504, an inner surface 506, and an outer surface 508. In some embodiments, the balloon 400 can be coaxially surrounded by the cover 500. For example, the inner surface 506 may substantially conform to an outer surface of the balloon 400, such that the balloon 400 and the cover 500 substantially comprise the same shape. In some embodiments, the cover 500 may be a different shape or configuration than the balloon 400.

In some embodiments, the cover 500 is also configured to elastically lengthen during radial expansion of the balloon 400. For example, during balloon expansion, the balloon 400 may lengthen longitudinally, imparting a longitudinal lengthening force on the cover 500. The cover 500 then, in turn, imparts a longitudinal lengthening force to the device 300, which may counteract any longitudinal foreshortening forces that may occur as a result of the device expanding. As used herein, “foreshortening” may be the percentage by which the length of the device 300 decreases when expanded from its unexpanded diameter to its expanded diameter or, in other terms, from its unexpanded configuration to its expanded configuration. In some embodiments, the device 300 may foreshorten less than 20 percent when used with the cover 500, as opposed to a system 100 without a cover 500.

The cover 500 can have a length 512 that is greater than the length 412 of the balloon 400. In some embodiments, the cover 500 fully covers the balloon 400 such that the first cover end 502 and the second cover end 504 extend beyond the first balloon end 502 and the second balloon end 504. In some embodiments, the cover 500 may then be axially compressed or scrunched at the first balloon end 402 and the second balloon end 404. In some embodiments, the cover 500 may only be compressed or scrunched at one end (e.g., either the first balloon end 402 or the second balloon end 404) or the cover 500 may not be compressed or scrunched at either end.

In some embodiments, the cover 500 is radially distensible up to a stop point that restricts, or in some instances resists, further expansion of the balloon 400 at a relatively constant inflation pressure. For example, the stop point may restrict or resist expansion of the balloon 400 past the nominal balloon diameter Dₙ or, in another example, past the maximum burst pressure diameter Dₘₐₓ of the balloon 400. In such examples, the cover 500 may reduce overexpansion or rapid unfurling of the balloon 400, which may protect and reduce trauma to the patient.

The cover 500 may comprise a polymer such as, for example, expanded fluoropolymers, such as expanded polytetrafluoroethylene (ePTFE), modified (e.g., densified) ePTFE, and expanded copolymers of PTFE. In some examples, the polymer can comprise a node and fibril microstructure and/or may be highly fibrillated (i.e., a non-woven web of fused fibrils). For example, the cover 500 may include serpentine fibrils. In some embodiments, the serpentine fibrils may allow the cover 500 to be both radially or circumferentially distensible and axially or longitudinally distensible. Therefore, the cover 500 can expand with the balloon 400 and does not limit expansion of the balloon 400 or cause uneven expansion of the balloon 400 and/or device 300.

In some embodiments, the cover 500 may include an elastomer such as, for example, PMVE-TFE (perfluoromethylvinyl ether-tetrafluoroethylene) copolymers, PAVE-TFE (perfluoro (alkyl vinyl ether)-tetrafluoroethylene) copolymers, silicones, and polyurethanes, among others. In some examples, the elastomer may help cause the serpentine fibrils to recover their shape, length, etc. after elongation and/or expansion of the cover 500. Examples of suitable covers and/or cover materials and/or stents may be found in U.S. publication number 2014/0172066 entitled “Medical Balloon Devices and Methods” filed on Dec. 18, 2013 by W. L. Gore & Associates, Inc. and U.S. publication number 2016/0143759 entitled “Balloon Expandable Endoprosthesis” filed on Nov. 24, 2015 by W. L. Gore & Associates, Inc.

In some embodiments, the cover 500 is a composite that includes an elastomer and an expanded, polytetrafluoroethylene matrix including serpentine fibrils. Non-limiting examples of suitable serpentine-fibril and elastomer composites can be found in U.S. publication number 2013/0184807 entitled “Articles Including Expanded Polytetrafluoroethylene Membranes with Serpentine Fibrils and Having a Discontinuous Fluoropolymer Layer Thereon, filed on Nov. 13, 2012 by W. L. Gore & Associates, Inc. The serpentine fibrils may be formed, or arranged in multiple directions (e.g., biaxial) or a single direction (i.e., uniaxial). For example, the serpentine fibrils can be in a longitudinal direction (along the longitudinal axis A_{L}) and a circumferential direction when the cover has been applied to the balloon 400. Use of such covers 500 can help facilitate compliance or stretch in the longitudinal and circumferential directions, thereby facilitating use of a cover 500 for multiple different balloon diameters. For example, a cover 500 could be used on a 5 mm balloon, a 6 mm balloon, a 7 mm balloon, a 8 mm balloon, a 9 mm balloon, a 10 mm balloon, a 11 mm balloon or larger or some interval or combination thereof.

In some examples, the cover 500 has a “stretch” characteristic corresponding to the amount the cover material (e.g., film matrix) is allowed to stretch without tearing after the cover 500 becomes taut. Such a “stretch” characteristic may be built into the cover 500 such that when the cover 500 is applied to a balloon 400 at 3 mm, for example, the cover 500 becomes taut at a larger diameter, 5 mm for example, but still accommodates another larger diameter, such as up to 10 mm, or 12 mm, or 14 mm or 16 mm or 18 m or more, for example, without tears. Although some examples of dimensions have been provided, any of a variety of dimensions is possible.

In some examples, the radial stretch, or stretchability of the cover 500 without exhibiting tearing can be 10%, 25%, 50%, 100%, 200%, 300%, 400% or more. Additionally, the cover 500 may accommodate lengthening of a balloon 400, or stretch in the longitudinal direction. For example, the balloon 400 may facilitate elongation in the longitudinal direction by 1 mm, 3 mm, 5 mm, 7 mm, 9 mm or more. In such examples, lengthening of the balloon 400 may be accommodated by longitudinal stretch of the cover 500. Longitudinal stretch of the cover may be 10%, 15%, 20% of an initial length of the cover 500 or more. The cover 500 may facilitate different amounts, or degrees of stretch in a longitudinal direction and a radial direction. In some examples, the cover 500 is applied to a deflated balloon 400 (e.g., while the balloon 400 is in a relaxed state) such that when the balloon 400 is expanded to its desired nominal diameter, the cover 500 transitions between a relaxed state and a stretched state. In other embodiments, the cover 500 is applied to the deflated balloon 400 such that the cover 500 is in a state when the balloon 400 is expanded to its desired nominal diameter.

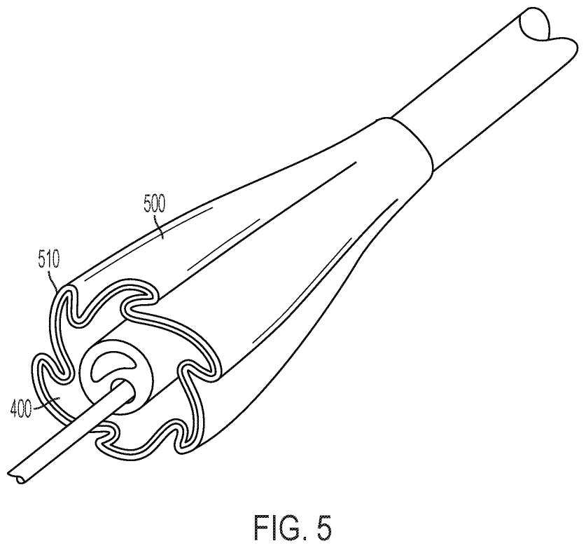

FIG. 5 is a cutaway view of a portion of the catheter system 100 showing a pleated and folded cover 500 and an interior of the balloon 400. As shown, in some examples the balloon 400 may be pleated, folded, or scrunched during compression. Additionally or alternatively, in some embodiments, the cover 500 may be folded, pleated, scrunched, or otherwise compressed.

In various embodiments, the cover 500 includes a plurality of pleats 510. The pleats 510 can include folds or inflection points in the material of the cover 500 extending generally along at least a portion of the length of the cover 500. The pleats 510 may assist with, or generally be a result of, compression of the balloon 400 and cover 500.

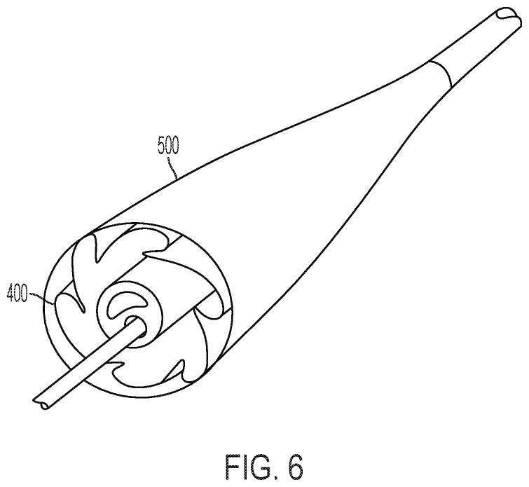

FIG. 6 is a cutaway view of a portion of the catheter system 100 showing a cover 500 having no pleats or folds and interior of the balloon 400 having pleats and folds, according to some embodiments. As shown, the balloon 400 may be folded, pleated, or scrunched while the cover 500 is free of pleats 510. Therefore, the cover 500 may be compressed without the use of folds, pleats, or scrunches. In such embodiments, the cover 500 may be taught while the balloon 400 has pleats and/or folds. As the balloon 400 is inflated and unfolds, the cover 500 may stretch to become more taut. In some embodiments, the cover may also have therapeutic treatments such as drug substances (e.g., heparin, antibiotics, and the like) applied to the cover surface. Such therapeutic treatments may promote healing, reduce tissue inflammation, reduce or inhibit infections, and/or promote various other therapeutic outcomes.

In some embodiments, the cover 500 may be folded, pleated, or scrunched while the balloon 400 is free of pleats or folds, or both the balloon 400 and cover 500 may have no pleats or folds.

The device shown in FIG. 6 is provided as an example of the various features of the device and, although the combination of those illustrated features is clearly within the scope of invention, that example and its illustration is not meant to suggest the inventive concepts provided herein are limited from fewer features, additional features, or alternative features to one or more of those features shown in FIG. 5. For example, in various embodiments, the balloon of the device shown in FIG. 6 may include the characteristics described with reference to FIG. 5. It should also be understood that the reverse is true as well. One or more of the components depicted in FIG. 5 can be employed in addition to, or as an alternative to components depicted in FIG. 6. For example, the cover and/or balloon of the device shown in FIG. 5 may be employed in connection with the cover and/or balloon of the device shown in FIG. 6.

### EXAMPLES

### Example 1: Elongation of Precursor Membrane

A biaxially expanded ePTFE membrane was obtained. The membrane had a thickness of approximately 0.007 mm and a density of approximately 0.18 g/cm². The matrix tensile strength was approximately 420 MPa in the direction of the fibrils and approximately 256 MPa in the opposite direction. When a tension force was applied in the direction of the fibrils, elongation of the membrane was approximately 74% at maximum loading. Elongation in the opposite direction was approximately 151% at maximum loading.

### Example 2: Effect of Heating Precursor Membrane on Elongation

A roll of precursor membrane, as described in Example 1, was restrained in a heated, uniaxial tenter frame. The membrane had an initial width of approximately 1,500 mm. The membrane was then fed into a heated chamber of the tenter frame, in which the temperature was set to about 300° C. The rails of the tenter frame were angled slightly inward to allow for membrane shrinkage to about 27% of its original width during heating. The final width of the membrane was approximately 400 mm.

The final membrane had a similar thickness of approximately 0.007 mm but a higher density of approximately 0.76 g/cm², as compared to the precursor membrane before heating. The matrix tensile strength in the direction of the fibrils decreased to approximately 238 MPa and approximately 90 MPa in the opposite direction. When a tension force was applied in the direction of the fibrils, elongation of the membrane was approximately 125% at maximum loading. Elongation in the opposite direction was approximately 620% at maximum loading. Therefore, it was concluded that heating the precursor membrane as described above resulted in a membrane capable of greater biaxial elongation and/or expansion, as compared to the precursor membrane before heating.

### Example 3: Preparation of Elastomeric Composite Film

A polyurethane elastomer (Tecothane® TT-107A) was dissolved in tetrahydrofuran to a concentration of about 5 wt. % in solution. The solution was then coated onto the precursor membrane of Example 2 using a slot die coating process. The weight percent of the elastomer within the composite film material was about 75 wt. %. After the elastomer was imbibed onto the membrane, the elastomeric composite film had a length of approximately 65 mm and a width of 114 mm. The thickness of the composite film material was approximately 0.014 mm.

A length of the elastomeric composite film was stretched by hand to an additional 78% of its original length. When stretched, the fibrils were observed to retain a serpentine shape. The tensile strength of the elastomeric composite film was approximately 104 MPa.

### Example 4: Preparation of a Balloon Having an 11.7 mm Nominal Diameter

A balloon having a working length of 82 cm and a diameter of approximately 11.7 mm at a nominal inflation pressure of 6 atm was obtained in a pleated and folded state. The balloon had a double wall thickness of less than about 0.07 mm (70 μm).

The elastomeric composite film of Example 3 was laid on a flat surface. When on the flat surface, the film had a composite section of polyurethane over ePTFE that measured approximately 40 mm by 114 mm and a non-composite section (without polyurethane) that measured approximately 28 mm by 114 mm. The composite film was then oriented such that its fibrils were oriented circumferentially around a 3.0 mm mandrel. The composite film was then wrapped circumferentially around the mandrel. The mandrel with the composite film was then heated to 190° C. for about three minutes. Each end of the composite film was attached to the balloon catheter in a pleated and folded state with an adhesive. The film was then cured.

### Example 5: Preparation of a Balloon Having a 7.0 mm Nominal Diameter

A balloon having a working length of about 17.5 mm and a diameter of 7.4 mm at a nominal inflation pressure of 6 atm was obtained. The balloon had a double wall thickness of less than about 0.060 mm (60 μm). The elastomeric composite film of Example 3 was placed on a flat surface. The film had a composite section of polyurethane over ePTFE of approximately 40 mm by 152 mm and a non-composite section (with no polyurethane) of approximately 28 mm by 152 mm. The composite film was then oriented such that its fibrils were oriented circumferentially around a 3.0 mm mandrel. The film was then wrapped circumferentially onto the mandrel. The mandrel with the composite film was then heated to 190° C. for about three minutes. The composite film was then cut to a width of 40 mm and each end of the composite film was attached to the catheter with an adhesive. The film was then cured.

### Example 6: Comparison of Nominal Inflation Pressures

Balloon 1 is an example of a balloon used in conventional catheter systems and was used as a controlled variable. Balloon 1 is a bilumen, nylon percutaneous transluminal angioplasty (PTA) balloon available from BMT Products. Balloons 2 and 3 were prepared as described above in Examples 4 and 5. Each balloon was maintained on an 80-cm catheter. The balloons were then inflated to a nominal inflation pressure diameter and the nominal inflation pressure was measured. Results are shown in Table 1 below.

TABLE 1

| | Balloon 1 | Balloon 2 | Balloon 3 |
|---|---|---|---|
| Nominal Device Diameter | 11.0 | 11.0 | 7.0 |
| (mm) | | | |
| Nominal Balloon Diameter | 11.0 | 11.7 | 7.4 |
| (mm) | | | |
| Balloon Working Length | 82 | 82 | 82 |
| (mm) | | | |
| Nominal Inflation Pressure | 11 atm at | 9 atm at | 11 atm at |
| (atm) | 11.0 mm | 11.0 mm | 7.0 mm |
| Rated Burst Pressure | 12 | 10 | 12 |
| (atm) | | | |
| Balloon Wall Thickness | 85 | 70 | 60 |
| (μm) | | | |
| Lumen Wall Thickness | 0.15 | 0.10 | 0.10 |
| (mm) | | | |
| Delivery Diameter (mm) | 2.81 | <2.58 | <2.25 |

As shown in Table 1, Balloon 1 had a smaller nominal balloon diameter and larger wall thickness than Balloon 2, though both Balloons 1 and 2 had the same length. Balloon 2 had a larger nominal balloon diameter and a smaller wall thickness. As shown, Balloon 2 had a smaller delivery diameter than Balloon 1, even though Balloon 2 had a larger nominal balloon diameter. Therefore, it was concluded that Balloon 2 was capable of achieving a lower profile in the delivery configuration as a result of an increased nominal balloon diameter and smaller balloon wall thickness as compared to Balloon 1.

As shown in Table 1, Balloon 2 required less pressure to inflate to a certain desired diameter than Balloon 1. For example, Balloon 2 required approximately 9 atm to inflate to the nominal inflation pressure diameter, which was less than Balloon 1, which required about 11 atm to inflate to the same diameter. Further, Balloon 2 had a rated burst pressure of 10 atm, while Balloon 1 had a rated burst pressure of 12 atm. Therefore, increasing the balloon nominal inflation pressure diameter allowed for an increased ratio of the rated burst pressure to the nominal inflation pressure, thus also increasing the safety factor. This increased safety factor allowed for a decrease in the balloon wall thickness and a lower profile when in the delivery configuration.

Balloon 3 was smaller than Balloon 2, having a smaller nominal balloon diameter as well as a smaller nominal device diameter. As expected, Balloon 3 showed similar characteristics to Balloon 2 and was capable of achieving an even smaller delivery diameter, as compared to both Balloon 2 and Balloon 1. Therefore, it was concluded that varying sizes of balloons exhibit similar, reduced profile characteristics.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the above described features.

1. A system for delivery of an implantable medical device, the system comprising:
an implantable medical device having a delivery diameter and a nominal device diameter, the implantable medical device capable of expanding from the delivery diameter to the nominal device diameter, wherein the nominal device diameter is larger than the delivery diameter; and
a delivery catheter comprising a balloon, wherein the implantable medical device is mounted on the balloon, and wherein the balloon has a nominal balloon diameter that is at least 3.5% larger than the nominal device diameter.

2. The system of claim 1, wherein the balloon has a nominal balloon diameter at a nominal inflation pressure of less than about 8 atm when the implantable medical device is mounted on the balloon.

3. The system of claim 1, wherein the balloon has a nominal balloon diameter that is from 3.5% to 10% larger than the nominal device diameter.

4. The system of claim 1, wherein the balloon has a nominal balloon diameter that is from 5% to 10% larger than the nominal device diameter.

5. The system of claim 1, wherein the balloon has a nominal balloon diameter from 4 mm to 15 mm.

6. The system of claim 1, wherein the balloon has a nominal balloon diameter of about 7.5 mm.

7. The system of claim 1, further comprising a cover that restricts expansion of the balloon disposed over at least a portion of the balloon.

8. The system of claim 7, wherein the cover comprises an elastomer.

9. The system of claim 7, wherein the cover is distensible in a radial and an axial direction.

10. The system of claim 7, wherein the cover comprises serpentine fibrils.

11. The system of claim 7, wherein the cover is radially distensible up to a stop point that restricts expansion of the balloon at a relatively constant inflation pressure.

12. A balloon catheter, comprising:
a catheter;
a balloon mounted on the catheter, the balloon having a nominal inflation pressure and a nominal balloon diameter; and
a medical device mounted on the balloon and having a nominal device diameter;
wherein the nominal balloon diameter of the balloon is at least about 5% greater than the nominal device diameter, and wherein the nominal inflation pressure of the balloon is about 6 atm when the medical device is mounted on the balloon.

13. A catheter system, comprising:
a balloon coupled to a catheter, the balloon having a rated burst pressure, and wherein the balloon has a double wall thickness less than about 60 μm; and
a medical device positioned along at least a portion of the balloon, the medical device having a nominal device diameter;
wherein a ratio of a pressure of the balloon at the nominal device diameter to the rated burst pressure of the balloon is less than about 0.8.

14. The catheter system of claim 13, wherein the balloon and the medical device are configured to be delivered through a diameter of less than about 2.5 mm when the medical device is positioned along at least a portion of the balloon.

15. A system for delivery of an implantable medical device, the system comprising:
a balloon having a length;
a cover disposed over at least a portion of the balloon; and
an implantable medical device disposed over at least a portion of the cover, wherein the cover elastically lengthens during radial expansion of the balloon from a compressed state to an expanded state and imparts a longitudinal lengthening force to the implantable medical device.

16. The system of claim 15, wherein the cover reduces foreshortening of the implantable medical device.

17. The system of claim 15, wherein the implantable medical device foreshortens less than 20% as compared to the length of the balloon when in a compressed state.

18. A method for assembling a catheter system, the method comprising:
selecting a balloon catheter with a balloon having a nominal balloon diameter that is at least 5% larger than a nominal device diameter of an implantable medical device;
mounting the implantable medical device over at least a portion of the balloon; and
compressing the implantable medical device and the balloon into a delivery diameter, wherein upon expansion of the balloon to the nominal device diameter, a nominal inflation pressure of the balloon is less than or equal to a rated burst pressure of the balloon.

19. The method of claim 18, further comprising: placing a cover over the balloon; and mounting the implantable medical device over the cover such that the cover imparts a longitudinal lengthening force to the medical device upon inflation of the balloon.

20. The method of claim 18, wherein the delivery diameter is from 1.5 mm to 3 mm.
